(19) United States
(12) Patent Application Publication
(10) Pub. No.: US 2022/0270705 A1
(43) Pub. Date: Aug. 25, 2022

(54) AUTOMATICALLY DESIGNING SELECTIVE MOLECULES

(71) Applicant: International Business Machines Corporation, Armonk, NY (US)

(72) Inventors: Enara C. Vijil, Millwood, NY (US); Payel Das, Yorktown Heights, NY (US); Inkit Padhi, White Plains, NY (US)

(21) Appl. No.: 17/185,148

(22) Filed: Feb. 25, 2021

(51) Int. Cl. G16B 15/30; G06N 20/00; G06N 3/02

(52) U.S. Cl. CPC G16B 15/30; G06N 20/00; G06N 3/02

(57) ABSTRACT

Generating a drug molecule design by training an attribute predictor model using an embedding of a first molecular data base, training a first machine learning model using the attribute predictor, yielding a second embedding of the first molecular data base, training a binding affinity model using a second molecular database and the second embedding of the first molecular database, and generating a molecule design according to the second embedding and the binding affinity model.

The following disclosure is submitted under 35 USC § 102(b)(1)(A): CHENTHAMARAKSHAN et al., “CogMol: Target-Specific and Selective Drug Design for COVID-19 Using Deep Generative Models”, arXiv:2004.01215v2 [cs.LG] 24 Jun. 2020, Preprint—Under review, 29 pages.

### BACKGROUND

The disclosure relates generally to the automatic designing of molecules. The disclosure relates particularly to automatically designing molecules having high target specificity and high target selectivity for novel targets.

Generative machine learning models enable the automated design of new molecules. Such models also enable the design of new molecules which bind to specific protein sequences.

### SUMMARY

The following presents a summary to provide a basic understanding of one or more embodiments of the disclosure. This summary is not intended to identify key or critical elements or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatuses and/or computer program products enable the generation of drug molecule designs.

Aspects of the invention disclose methods, systems and computer readable media associated with generating a molecule design by training a binding affinity model using a first molecular database and an embedding of a second molecular database, and generating a molecule design according to the embedding and the binding affinity model.

### BRIEF DESCRIPTION OF THE DRAWINGS

Through the more detailed description of some embodiments of the present disclosure in the accompanying drawings, the above and other objects, features and advantages of the present disclosure will become more apparent, wherein the same reference generally refers to the same components in the embodiments of the present disclosure.

FIG. 1 provides a schematic illustration of a computing environment, according to an embodiment of the invention.

FIG. 2 provides a flowchart depicting an operational sequence, according to an embodiment of the invention.

FIG. 3 depicts a cloud computing environment, according to an embodiment of the invention.

FIG. 4 depicts abstraction model layers, according to an embodiment of the invention.

### DETAILED DESCRIPTION

Some embodiments will be described in more detail with reference to the accompanying drawings, in which the embodiments of the present disclosure have been illustrated. However, the present disclosure can be implemented in various manners, and thus should not be construed to be limited to the embodiments disclosed herein.

In an embodiment, one or more components of the system can employ hardware and/or software to solve problems that are highly technical in nature (e.g., training a first machine learning model using a first molecular database, training a binding affinity model using the embedding of the first molecular database and a second molecular database, generating a drug molecule design according to the embedding and the binding affinity model, etc.). These solutions are not abstract and cannot be performed as a set of mental acts by a human due to the processing capabilities needed to facilitate generating drug molecule designs, for example. Further, some of the processes performed may be performed by a specialized computer for carrying out defined tasks related to generating molecule designs. For example, a specialized computer can be employed to carry out tasks related to the generation of molecule designs, or the like.

Generating novel molecule designs, including novel drug molecule designs, is a daunting task that aims to create new molecules (or optimize known molecules) with multiple desirable properties that often compete and tightly interact with each other. For example, optimal drug molecules should have binding affinity to the target protein of interest (target specificity), have low binding affinity to other targets (off-target selectivity), be easy to synthesize, and also exhibit high drug likeliness (QED). This makes drug discovery a costly (2-3 billion USD) and time-consuming process (more than a decade) with a low success rate (<10%).

Traditional in silico molecule design and screening methods rely on rational design methods that need physics-based simulations, heuristic search algorithms, and considerable domain knowledge. However, optimizing over the discrete, unstructured, and sparse molecular space remains an intrinsically difficult challenge. Therefore, there is interest in developing automated machine learning techniques to efficiently discover sizeable numbers of plausible, diverse, and novel candidate molecules in the vast (10²³-10⁶⁰) space of molecules. Bayesian optimization, reinforcement learning, and gradient based optimization methods have been proposed for automating drug molecule design with desired properties (e.g., high drug-likeliness, synthetic accessibility, or solubility). These methods either optimize directly on the high-dimensional input space or on the low dimensional representation learned using a latent variable model such as a probabilistic autoencoder.

One crucial aspect of designing drug candidates is to account for the right context, e.g., protein, gene, metabolic or disease pathway information. For example, in target protein-specific drug design, the goal is to generate molecules with high binding affinity to a specific target protein. This requires fine-tuning a generative model on a small library of ligands to enable target-specific sampling. For novel or unrelated proteins, such as the SARS-CoV-2 viral proteins involved in the recent COVID-19 pandemic, binding affinity data is unavailable. At the same time, these novel target proteins are not related to the proteins in existing binding affinity databases. Thus, handling novel targets in the current generative frameworks becomes non-trivial.

Designing drug candidates for novel targets gets even more challenging, as the drug designed for the novel target can bind to other undesired targets. Small molecule drugs have been shown to bind on average to a minimum of 6-11 distinct targets in addition to their intended target. This molecular “promiscuity” of drugs causes unintended therapeutic effects or multiple drug—target interactions leading to off-target toxicities and decreased effectiveness. Accounting for this important aspect of off-target selectivity becomes non-trivial if the generative model is trained only on a small ligand library optimized for a single target or only on good binder molecules for a limited set of targets.

Generative machine learning models enable the design of new drug molecules which bind to specific proteins. Such models do not account for the selectivity of the new drug molecules. As used herein, selectivity refers to the relative binding affinity between a new drug molecule, the target protein sequence, and non-target protein sequences, or the excess binding affinity of a molecule to the target protein over the average binding affinity to a random selection of non-target proteins. A selective drug molecule has a high binding affinity to the target protein sequence and a relatively low binding affinity to non-target protein sequences. A non-selective new drug molecule has similar binding affinities to the target protein sequence and to non-target protein sequences. The similar binding affinities result in the non-selective drug molecule indiscriminately binding to a large number of protein sequences. The non-selective drug molecule has a higher likelihood of being toxic, due to this indiscriminate binding. Disclosed embodiments enable the automated design of new drug molecules having a high selectivity for the target protein sequence relative to other protein sequences.

Disclosed embodiments include the use of a machine learning model, such as a variational autoencoder (VAE). In an embodiment, the method initially trains the VAE using unsupervised learning and a first database of molecules such as a set of molecules described using simplified molecular line-entry system (SMILES) representations. In an embodiment, the method uses molecule representations from the recursively named, ZINC is not commercial (ZINC) database to train the VAE. Training the VAE yields an embedding of the molecule data encompassed by the database, where the embedding includes latent relationships in the data. The method utilizes the embedding including the latent relationships in training one or more molecule attribute regressor function predictors, such as predictor models for synthetic accessibility (SA), the partition coefficient between octanol and water (log P), and a quantitative estimate of drug-likeliness (QED), predictors for molecules using labeled data from the molecule database.

In an embodiment, the method continues and augments the training of the VAE. The method includes the one or more of the molecule attribute regressor functions in training the VAE, yielding a second embedding and set of latent relationships including the relationships associated with the predicted molecule attributes. In this embodiment, the method utilizes molecule representations from the BINDINGDB as part of the training data, rather than the ZINC molecule representations.

In an embodiment, the method utilizes the latent features of the molecules from the trained VAE embedding and a large corpus of protein sequence embeddings to train a binding affinity model to predict on-target and off-target molecule-protein affinities. The method may utilize the embedding of the original VAE or the embedding of the augmented VAE in training the binding affinity model. The affinities enable predictions of target specificity (the binding affinity between a molecule and a target protein sequence) as well as target selectivity (the relative biding affinity between the molecule and target protein sequence compared to the binding affinity between the molecule and other non-target protein sequences. A high molecule—target binding affinity indicates high target specificity and a high molecule-target affinity relative to molecule off-target protein sequences affinities, indicates a high target—off target selectivity.

In this embodiment, training the binding affinity model includes the use of pre-trained protein sequence embeddings learned using an unlabeled corpus of protein sequences such as 24 million UNIPROT protein sequences from the UNIREF50 database, including sequence structural and functional relationships. The use of the large corpus of protein sequence data enables a more generalized model capable of generating new molecule designs for novel protein sequences. In this embodiment, training the binding affinity model further includes labeled training data, such as IC50-labeled compound-protein binding data from BINDINGDB, including pIC50, or −log(IC50) data. Training the binding affinity model may further include the use of a language model, such as GLOVE, WORD2VEC, etc., to derive data labels from the protein sequence data itself to support self-supervised learning of the binding affinity model. (Note: the terms “ZINC”, “BINDINGDB”, “UNIPROT”, “GLOVE”, “WORD2VEC”, and “UNIREF50”, may be subject to trademark rights in various jurisdictions throughout the world and are used here only in reference to the products or services properly denominated by the marks to the extent that such trademark rights may exist.)

In this embodiment, the trained binding affinity model receives a continuous representation of a target protein sequence. In this embodiment, the continuous representation is a vector in a statistical computing environment such as R, that captures the relationships between protein sequences such that proteins that are similar are closer together in this continuous space. The binding affinity model also receives a latent embedding representation of a prospective drug molecule from the augmented VAE model and predicts a binding affinity between the compound and the protein.

In an embodiment, the method uses pre-trained protein embeddings, such as UNIPROT data available from the UNIREF50 database, to initialize network weights for protein sequences. In an embodiment, the method provides a SMILES representation of the prospective compound together with the UNIREF50 representation of the target protein to the binding affinity model. The binding affinity model provides a prediction of the binding energy between the prospective compound and the target protein sequence.

The combination of the VAE and the binding affinity models yield molecule designs selected for target specificity, off-target selectivity, as well as SA, log P, and/or QED. Subsequent to the training of the machine learning model augmented with the molecule attribute predictor(s), and the binding affinity model, the method provides the trained models for use in designing/generating molecule designs according to specified target protein sequences, target specificities and target selectivities. The method receives a request for a design, the request including one or more target protein sequence representations and associated target specificity value ranges and target selectivity value ranges. The request may further include molecule attribute value ranges for log P, SA, and QED values for the generated molecular designs. The method provides the design request details as inputs to the original or augmented VAE model. The original VAE provides drug designs satisfying the target specificity and target selectivity criteria. The augmented VAE model provides one or more drug molecule designs which also satisfy the molecular attribute design criteria. The output designs and design request target protein representation(s) are provided as inputs to the binding affinity model. The binding affinity model provides compound-protein binding affinity value predictions for each combination of drug molecule design/target protein sequence combination. In an embodiment, the method limits the outputs of the binding affinity model to those compound—protein combinations which satisfy the target specificity design criteria. In an embodiment, the method may further determine binding affinities for one or more of the drug molecule designs and a set of one or more non-target proteins, in order to evaluate the target selectivity of the drug molecule design. In this embodiment, the method evaluates the relative binding affinity of a drug molecule design with each of the target and non-target proteins. The method outputs and provides those drug molecule designs exceeding a selectivity threshold difference between the target protein binding affinity and the non-target binding affinities.

In an embodiment, the method generates molecule designs which concurrently satisfy design criteria for a high binding affinity to a selected novel protein sequence, a high drug likeliness, and a high off-target selectivity. In this embodiment, the method performs conditional drug molecule design generation using conditional latent space sampling (CLaSS). CLaSS leverages the attribute predictor functions of the augmented VAE trained on the latent features of the original VAE.

CLaSS uses a rejection sampling scheme to generate samples with desired attributes from a density model of the latent space. Since the goal is to sample conditionally p(x|a), where a∈Rⁿ=[a₁,a₂, . . . ,aₙ], a set of independent attributes, CLaSS approaches this task through conditional sampling in latent space: p(x|a)=Ez[p(z|a)p(x|z)]≤Ez[p\hat{ }_{ξ}(z|a)p_{θ}(x|z)], where p\hat{ }_{ξ}(z|a) uses rejection sampling from parametric approximations to p(z|a). The method approximates the term p\hat{ }_{ξ}(z|a) using a density model Q_{ξ}(z), such as a Gaussian mixture model and per-attribute classifier model q_{ξ}(aᵢ|z). The method approaches the approximation task by using Bayes' rule and then conditional independence of the attributes. The method then performs rejection sampling through the proposal distribution: g(z)=Q_{ξ}(z) that can be directly sampled. Since multiple attribute constraints are imposed for the sampling, the acceptance probability is equal to the product of the attribute predictors' scores, while sampling from explicit density Q_{ξ}(z). As long as there is a region in z space where Q_{ξ}(z)>0 and probabilities from all predictors are >0, samples will be accepted in this scheme. Consequently, CLaSS can sample from the targeted region of the autoencoder latent space, which was trained unsupervised. Learning to control for one or more attribute(s) in CLaSS is computationally efficient, as it does not require a surrogate model or policy learning, nor does it add complicated loss terms to the original objective.

In an exemplary embodiment, the method evaluates molecular toxicity or side effect testing using a multitask deep neural network (MT-DNN) for binary (yes/no) toxicity prediction as an early screening tool to prioritize the testing of molecules that are less likely to be harmful and to speed up the process of finding a therapeutic. The method uses MT-DNN to predict the toxicity of 12 in vitro endpoints from the Tox21 challenge. The method also predicts whether the generated molecules will fail clinical trials, using ClinTox data.

In this exemplary embodiment, the method further screens generated molecules for target affinity and selectivity using an x-level binding affinity predictor. To investigate the possible binding modes of the generated molecules with the target protein structure, the method performs five independent runs of blind docking of the generated achiral molecules with the target structure using Autodock Vina. To evaluate the synthetic accessibility, the method analyzes generated molecules using a retrosynthetic algorithm based on the Molecular Transformer trained on patent chemical reaction data.

### EXAMPLE

Disclosed methods were used to generate drug molecules having high affinity for three protein sequences of the SARS Cov-2 S protein, NSP9 Replicase (NSP9), Main Protease (M^{pro}), and the Receptor Binding Domain (RBD), the method screened the generated molecules for high drug likeliness, and high selectivity to the three protein sequences. The majority of the generated molecules were chemically valid (90%), unique (99%), passed relevant filters (95%), and showed a slightly higher diversity.

For this example, the novelty distributions, as estimated using the Tanimoto Similarity between molecular fingerprints, of the generated molecules with respect to both the PubChem database and the training database of size ˜1.9 M, indicate that the likelihood of generating molecules with a novelty value of 0 is ≤2%. With respect to the larger PubChem database consisting of ˜103 M molecules, the majority of which were not included in model training database, the percentage of generated molecules with a novelty value of 0 is 9.5%, 3.7%, and 8.3% for generated molecules regarding M^{pro}, RBD, and NSP9, respectively. Higher FCD of those generated molecules with respect to test scaffolds in ZINC/BINDINGDB also indicates novel chemical scaffolds among the generated molecules.

Table 1 illustrates a higher proportion of molecules with desired attributes in the set accepted in CLaSS, when compared to a randomly sampled set, implying that CLaSS does generate a more optimal set than random sampling from the latent space, and the success depends on the target context. In an embodiment, around one thousand method-generated molecules were selected according to design criteria of binding affinity 0.6, QED>0.4, SA<0.4, number of toxic endpoints<2, log P<5, and an average molecular weight (Mw)<500 daltons. for each target.

TABLE 1

| | | | Aff > 0.5 | | Aff > 0.5 QED > 0.8 | |
|---|---|---|---|---|---|---|
| Target | Aff > 0.5 | | QED > 0.8 | | Sel > 0.5 | |
| sequence | ClaSS | Random | CLaSS | Random | CLaSS | Random |
| NSP9 | 0.567 | 0.355 | 0.45 | 0.211 | 0.069 | 0.007 |
| RBD | 0.546 | 0.369 | 0.429 | 0.217 | 0.09 | 0.009 |
| Mpro | 0.603 | 0.366 | 0.472 | 0.216 | 0.104 | 0.011 |

For the example, disclosed methods also identify PubChem Molecules with Potential Anti-COVID Activity. Only nineteen, five, and fifteen, of the generated molecules match exactly with an existing SMILES string in PubChem, for M^{pro}, RBD, and NSP9, respectively. Some of these SMILES are reported with biological activity in PubChem, as shown in Table 2. For example, the molecule with PubChem Compound ID (CID) 76332092 is a known Plasmepsin-2 and Plasmepsin-4 inhibitor and has also shown antimalarial activity against chloroquine-sensitive Plasmodium falciparum.

Table 2 illustrates the predicted affinity, docking energy, public compound ID (CID), and reported biological activity, for drug molecule designs generated using SMILES molecule representations rather than the augmented VAE latent molecule embedding.

TABLE 2

| | | Docking | | |
|---|---|---|---|---|
| Target | Pred. Affinity | energy | CID | Biological activity |
| | 6.51 | −7.7 | 12042753 | Antagonist of rat mGluR |
| NSP9 | 7.06 | −5.6 | 44397285 | Active to human S6 |
| | | | | kinase |
| Dimer | 7.18 | −6.4 | 10570770 | Matrix metalloproteinase |
| | | | | inhibitor |
| Main | 7.24 | −6.1 | 10608757 | Dihydrofolate reductase |
| Protease | 6.91 | −6.9 | 872399 | inhibitor Shiga toxin |
| | | | | inhibitor |
| RBD | 7.82 | −7.5 | 76332092 | Plasmepsin inhibitor |

Table 3 summarizes docking with the target protein sequence evaluation results. In the best (lowest BFE) docking pose, 87%, 91%, and 95% of generated molecules show a minimum BFE of <−6 kcal/mol for NSP9 dimer, M^{pro}, and RBD, respectively. For each target, each molecule was classified by its binding location, fitting the geometric centers of docked molecules drawn from a larger set of 875K samples to a mixture of 4, 5, and 6 Gaussian models, respectively (see Supp. Mat. H). The data also indicates the average and minimum BFE, as well as the fraction of generated molecules with a BFE of <−6 kcal/mol for each cluster (Table 3). Results show that even though CLaSS used only target sequence information for controlled generation, generated molecules do identify the relevant and known druggable binding pockets within the 3D target structure and bind to those favorably.

TABLE 3

| | | Size | E | Min | Low |
|---|---|---|---|---|---|
| Target | | (%) | (kcal/mol) | (kcal/mol) | energy (%) |
| NSP9 Dimer | cluster 0 | 67 | −6.8 ± 0.7 | −8.6 | 88 |
| (87%) | cluster 1 | 22 | −6.9 ± 0.9 | −8.8 | 85 |
| Main Protease | cluster 0 | 76 | −7.2 ± 0.8 | −9.5 | 93 |
| (91%) | cluster 1 | 18 | −6.9 ± 0.8 | −9.2 | 86 |
| RBD | cluster 0 | 30 | −6.9 ± 0.6 | −8.3 | 93 |
| (95%) | cluster 1 | 36 | −7.2 ± 0.6 | −9.1 | 97 |

Human HDAC1 plays key role in eukaryotic gene expression and is implicated in cancer. Though it is present in BINDINGDB, there are only a handful of molecules with high QED and high pIC50. Disclosed methods were applied to generate optimal ligands targeting HDAC1. Table 4 shows that method-generated molecules comprise a larger proportion of molecules satisfying high pIC50 and QED criteria, implying disclosed methods can discover novel and optimal molecules even in a low-data regime.

TABLE 4

| | Total | | pIC50 > 6 and | pIC50 > 7 and |
|---|---|---|---|---|
| Dataset | Molecules | QED > 0.8 | QED > 0.8 | QED > 0.8 |
| Train Set | 2253 | 43 (1.9%) | 9 (0.39%) | 1 (0.04%) |
| Generated | 1388 | 188 (13.6%) | 89 (6.42%) | 32 (2.3%) |

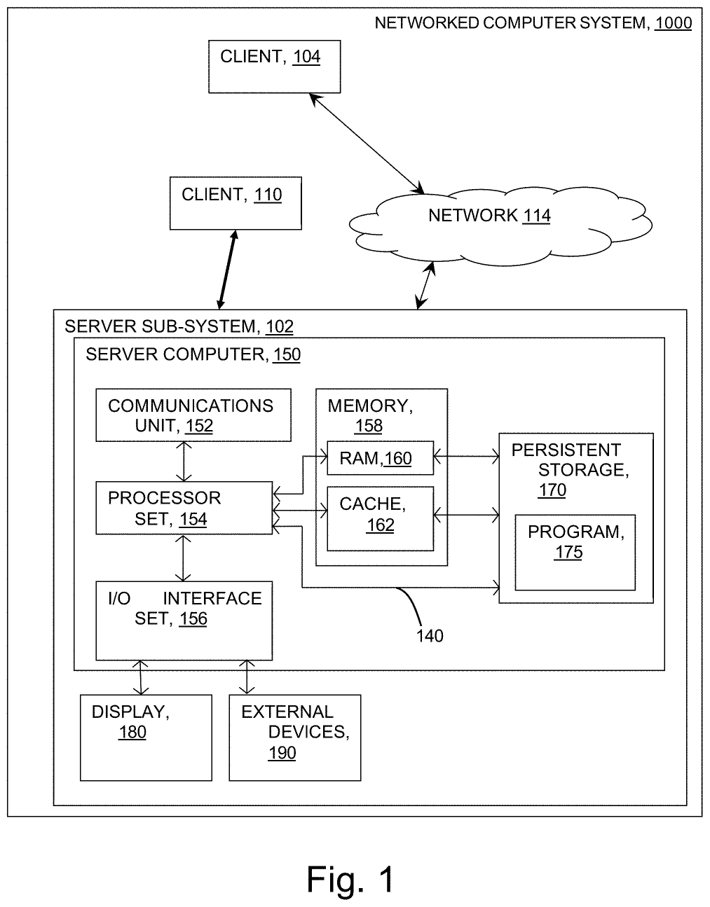

FIG. 1 provides a schematic illustration of exemplary network resources associated with practicing the disclosed inventions. The inventions may be practiced in the processors of any of the disclosed elements which process an instruction stream. As shown in the figure, a networked Client device 110 connects wirelessly to server sub-system 102. Client device 104 connects wirelessly to server sub-system 102 via network 114. Client devices 104 and 110 comprise drug molecule design application program (not shown) together with sufficient computing resource (processor, memory, network communications hardware) to execute the program. As shown in FIG. 1, server sub-system 102 comprises a server computer 150. FIG. 1 depicts a block diagram of components of server computer 150 within a networked computer system 1000, in accordance with an embodiment of the present invention. It should be appreciated that FIG. 1 provides only an illustration of one implementation and does not imply any limitations with regard to the environments in which different embodiments can be implemented. Many modifications to the depicted environment can be made.

Server computer 150 can include processor(s) 154, memory 158, persistent storage 170, communications unit 152, input/output (I/O) interface(s) 156 and communications fabric 140. Communications fabric 140 provides communications between cache 162, memory 158, persistent storage 170, communications unit 152, and input/output (I/O) interface(s) 156. Communications fabric 140 can be implemented with any architecture designed for passing data and/or control information between processors (such as microprocessors, communications and network processors, etc.), system memory, peripheral devices, and any other hardware components within a system. For example, communications fabric 140 can be implemented with one or more buses.

Memory 158 and persistent storage 170 are computer readable storage media. In this embodiment, memory 158 includes random access memory (RAM) 160. In general, memory 158 can include any suitable volatile or non-volatile computer readable storage media. Cache 162 is a fast memory that enhances the performance of processor(s) 154 by holding recently accessed data, and data near recently accessed data, from memory 158.

Program instructions and data used to practice embodiments of the present invention, e.g., the drug molecule design program 175, are stored in persistent storage 170 for execution and/or access by one or more of the respective processor(s) 154 of server computer 150 via cache 162. In this embodiment, persistent storage 170 includes a magnetic hard disk drive. Alternatively, or in addition to a magnetic hard disk drive, persistent storage 170 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 170 may also be removable. For example, a removable hard drive may be used for persistent storage 170. Other examples include optical and magnetic disks, thumb drives, and smart cards that are inserted into a drive for transfer onto another computer readable storage medium that is also part of persistent storage 170.

Communications unit 152, in these examples, provides for communications with other data processing systems or devices, including resources of client computing devices 104, and 110. In these examples, communications unit 152 includes one or more network interface cards. Communications unit 152 may provide communications through the use of either or both physical and wireless communications links. Software distribution programs, and other programs and data used for implementation of the present invention, may be downloaded to persistent storage 170 of server computer 150 through communications unit 152.

I/O interface(s) 156 allows for input and output of data with other devices that may be connected to server computer 150. For example, I/O interface(s) 156 may provide a connection to external device(s) 190 such as a keyboard, a keypad, a touch screen, a microphone, a digital camera, and/or some other suitable input device. External device(s) 190 can also include portable computer readable storage media such as, for example, thumb drives, portable optical or magnetic disks, and memory cards. Software and data used to practice embodiments of the present invention, e.g., drug molecule design program 175 on server computer 150, can be stored on such portable computer readable storage media and can be loaded onto persistent storage 170 via I/O interface(s) 156. I/O interface(s) 156 also connect to a display 180.

Display 180 provides a mechanism to display data to a user and may be, for example, a computer monitor. Display 180 can also function as a touch screen, such as a display of a tablet computer.

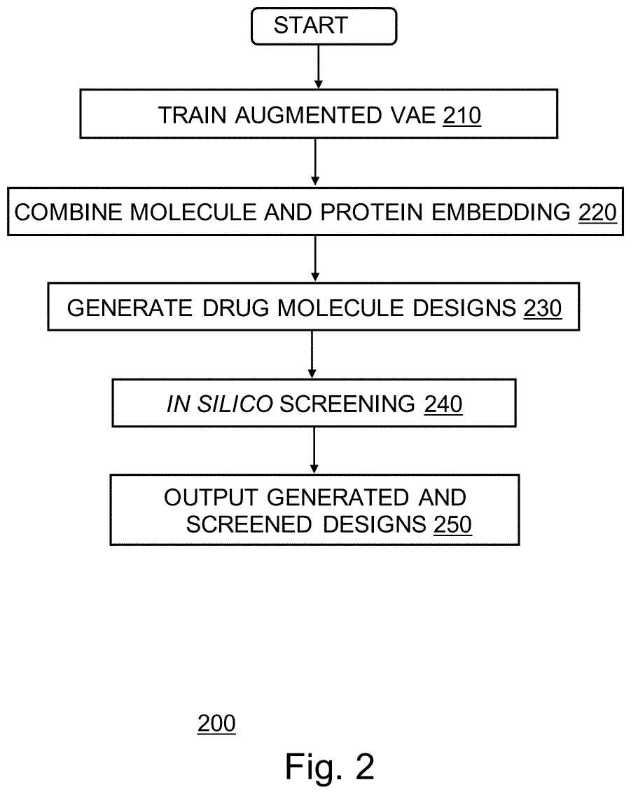

FIG. 2 provides a flowchart 200, illustrating exemplary activities associated with the practice of the disclosure. After program start, at block 210, molecule SMILES representations from ZINC and/or BINDINGDB, are used in training an augmented VAE, including one or more molecule attribute functions. The training of the VAE yields an embedding of the latent relationships in the molecule data training data set. In an embodiment, the method trains the VAE without the addition of the attribute predictor functions yielding a different embedding of the latent data relationships. At block 220, the embedding from block 210 combines with protein sequence embedding, such as UNIPROT data, for controlled generation of drug molecule designs according to provided drug design criteria—target protein and drug molecule attribute values, yielding generated drug molecule designs at block 230. In an embodiment, drug molecule design criteria include only target selectivity and target specificity criteria. In this embodiment, the latent embedding from the un-augmented VAE combines with the binding affinity model to generate drug molecule designs. Generated drug molecule designs undergo in silico screening at block 240, including toxicity, synthetic accessibility screening, and target protein sequence docking. The in silico screening yields the final set of generated and screened drug molecules at 250.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

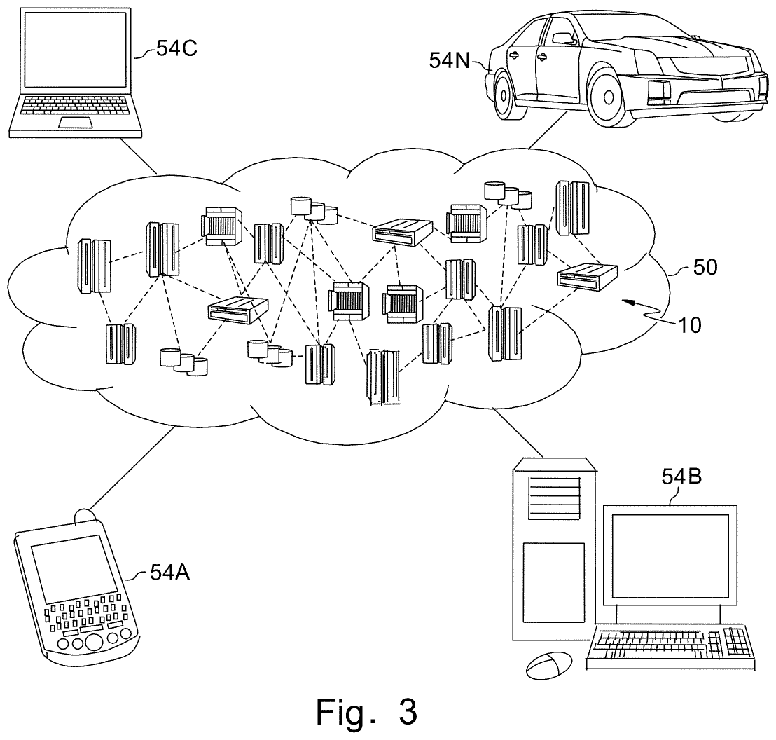

Referring now to FIG. 3, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 3 are intended to be illustrative only and that computing nodes 10 and cloud computing environment 50 can communicate with any type of computerized device over any type of network and/or network addressable connection (e.g., using a web browser).

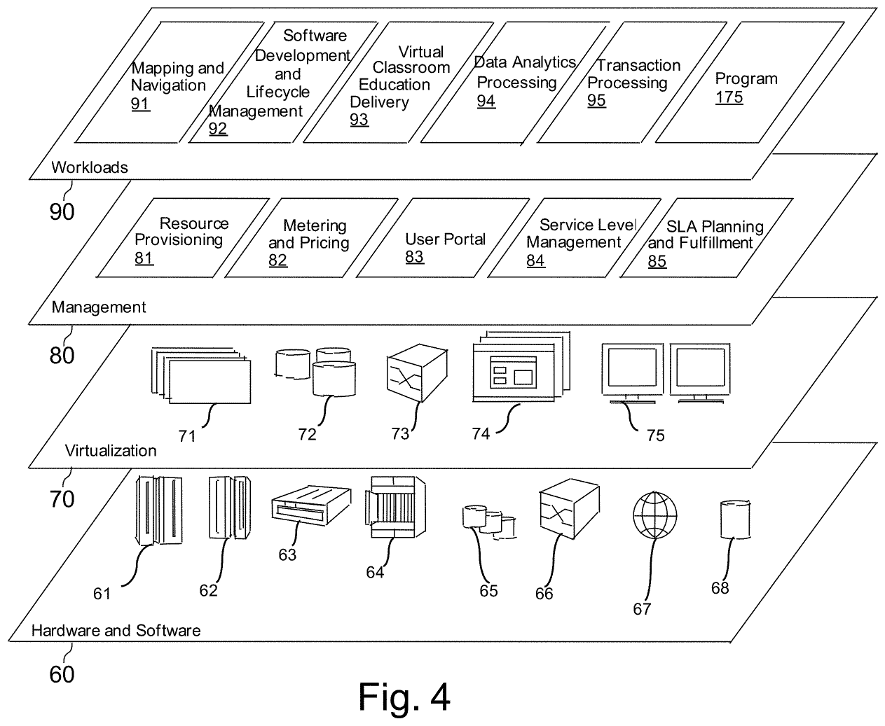

Referring now to FIG. 4, a set of functional abstraction layers provided by cloud computing environment 50 (FIG. 3) is shown. It should be understood in advance that the components, layers, and functions shown in FIG. 4 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and drug molecule design program 175.

The present invention may be a system, a method, and/or a computer program product at any possible technical detail level of integration. The invention may be beneficially practiced in any system, single or parallel, which processes an instruction stream. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, or computer readable storage device, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions collectively stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer implemented method for generating a molecule design, the method comprising:
training, by one or more computer processors, an attribute predictor model using an embedding of a first molecular database;
training, by the one or more computer processors, a first machine learning model using the attribute predictor, yielding a second embedding of the first molecular database;
training, by the one or more computer processors, a binding affinity model using a second molecular database and the second embedding of the first molecular database; and
generating, by the one or more computer processors, a molecule design according to the second embedding and the binding affinity model.

2. The computer implemented method according to claim 1, wherein training the attribute predictor model comprises a quantitative estimate of drug-likeliness.

3. The computer implemented method according to claim 1, wherein the first machine learning model comprises a machine learning model selected from the group consisting of a variational autoencoder, a generative neural network, and a reinforcement learning model.

4. The computer implemented method according to claim 1, further comprising:
receiving, by the one or more computer processors, a request for a molecule design, the request including a selective affinity for a target, and a specificity for the target;
providing, by the one or more computer processors, chemical properties of the target to the attribute predictor model and the binding affinity model; and
generating, by the one or more computer processors, a molecule design according to the chemical properties of the target, the attribute predictor model, and the binding affinity model.

5. The computer implemented method according to claim 4, wherein the target comprises a protein sequence.

6. The computer implemented method according to claim 4 wherein the molecule design has the selective affinity for the target and the specificity for the target.

7. The computer implemented method according to claim 1, wherein the attribute predictor model comprises a synthetic accessibility predictor.

8. A computer program product for generating a molecule design, the computer program product comprising one or more computer readable storage devices and collectively stored program instructions on the one or more computer readable storage devices, the stored program instructions comprising:
training an attribute predictor model using an embedding of a first molecular database;
training a first machine learning model using the attribute predictor, yielding a second embedding of the first molecular database;
training a binding affinity model using a second molecular database and the second embedding of the first molecular database; and
generating a molecule design according to the second embedding and the binding affinity model.

9. The computer program product according to claim 8, wherein training the attribute predictor model comprises a quantitative estimate of drug-likeliness.

10. The computer program product according to claim 8, wherein the first machine learning model comprises a machine learning model selected from the group consisting of a variational autoencoder, a generative neural network, and a reinforcement learning model.

11. The computer program product according to claim 8, the stored program instructions further comprising:
program instructions to receive a request for a molecule design, the request including a selective affinity for a target, and a specificity for the target;
program instructions to provide chemical properties of the target to the attribute predictor model and the binding affinity model; and
program instructions to generate a molecule design according to the chemical properties of the target, the attribute predictor model, and the binding affinity model.

12. The computer program product according to claim 11, wherein the target comprises a protein sequence.

13. The computer program product according to claim 11, wherein the molecule design has the selective affinity for the target and the specificity for the target.

14. The computer program product according to claim 8, wherein the attribute predictor model comprises a synthetic accessibility predictor.

15. A computer system for generating a molecule design, the computer system comprising:
one or more computer processors;
one or more computer readable storage devices; and
stored program instructions on the one or more computer readable storage devices for execution by the one or more computer processors, the stored program instructions comprising:
training an attribute predictor model using an embedding of a first molecular database;
training a first machine learning model using the attribute predictor, yielding a second embedding of the first molecular database;
training a binding affinity model using a second molecular database and the second embedding of the first molecular database; and
generating a molecule design according to the second embedding and the binding affinity model.

16. The computer system according to claim 15, wherein training the attribute predictor model comprises a quantitative estimate of drug-likeliness.

17. The computer system according to claim 15, wherein the first machine learning model comprises a machine learning model selected from the group consisting of a variational autoencoder, a generative neural network, and a reinforcement learning model.

18. The computer system according to claim 15, the stored program instructions further comprising:
program instructions to receive a request for a molecule design, the request including a selective affinity for a target, and a specificity for the target;
program instructions to provide chemical properties of the target to the attribute predictor model and the binding affinity model; and
program instructions to generate a molecule design according to the chemical properties of the target, the attribute predictor model, and the binding affinity model.

19. The computer system according to claim 18, wherein the target comprises a protein sequence.

20. The computer system according to claim 18, wherein the molecule design has the selective affinity for the target and the specificity for the target.
